(19) United States
(12) Patent Application Publication
(10) Pub. No.: US 2024/0115601 A1
(43) Pub. Date: Apr. 11, 2024

(54) NOVEL THERAPEUTIC PRODUCTS AND ITS METHOD OF PREPARATION

(71) Applicant: Senthilkumar NATESAN, Ahmedabad (IN)

(72) Inventor: Senthilkumar NATESAN, Ahmedabad (IN)

(21) Appl. No.: 18/546,274

(22) PCT Filed: Feb. 14, 2022
(86) PCT No.: PCT/IB2022/051280

§ 371 Date: Aug. 13, 2023

(30) Foreign Application Priority Data
Feb. 15, 2021 (IN) 202121006270

(51) Int. Cl. A61K 35/15; A61K 9/06

(52) U.S. Cl. CPC A61K 35/15; A61K 9/06

(57) ABSTRACT

The present invention provides novel therapeutic products which are used for treating chronic non healing wounds. The present invention is related to novel therapeutic products generated from peripheral blood mononuclear cells. The present invention also provides a process of preparing the therapeutic products, an aqueous suspension for Injection, gel and patch generated from peripheral blood cells in vitro having wound healing properties.

### FIELD OF THE INVENTION

The present invention is in the field of novel therapeutic products generated from peripheral blood mononuclear cells. The present invention also related to process of preparing the therapeutic products, an aqueous suspension for Injection, gel and patch generated from peripheral blood cells in vitro having wound healing properties.

### BACKGROUND OF THE INVENTION

A wound results from damage or disruption to normal anatomical structure and function. This can range from a simple break in the epithelial integrity of the skin to deeper, subcutaneous tissue with damage to other structures such as tendons, muscles, vessels, nerves, parenchymal organs and even bone. Irrespective of the cause and form, wounding damages and disrupts the local tissue environment. Wounds are classified according to various criteria. Among these, time plays an important role in injury management and wound repair. Thus, wounds can be clinically categorized as acute or chronic based on their time frame of healing.

Acute wounds are wounds that repair themselves and that proceed normally by following a timely and orderly healing pathway with both functional and anatomical restoration as the end result, are classified as acute wounds. Typically, healing of acute wounds range from 5-10 days or within 30 days. Acute wounds can be acquired, for example, as a result of traumatic loss of tissue or as a result of a surgical procedure. Acute wound healing is a well-organized process leading to predictable tissue repair where platelets, keratinocytes, immune surveillance cells, microvascular cells and fibroblasts play key role in the restoration of tissue integrity. Chronic wounds are wounds that fail to progress through the normal stages of healing and cannot be repaired in an orderly and timely manner. The healing process is incomplete and disturbed by various factors that prolong one or more stages in the phases of hemostasis, inflammation, proliferation or remodeling. These factors include infection, tissue hypoxia, necrosis, exudate and excess levels of inflammatory cytokines. Chronic wounds can be classified into vascular ulcers (eg, venous and arterial ulcers), pressure ulcers and diabetic ulcers. Some common features shared by each of these include a prolonged or excessive inflammatory phase, persistent infections, formation of drug-resistant infection. Diabetic foot ulcer and chronic non-healing wounds are major health problem, which occurs around 15% of the diabetes patients due to impaired wound healing and persistent inflammatory response. Diabetic foot is one of the most significant and devastating complications of diabetes, which is characterized by ulceration of the lower limb with neuropathy and/or peripheral arterial disease. The condition is more frequent in older patients. It is also the major cause of diabetes associated amputation, which required prolonged hospitalization and management. Mortality following amputation is 50% and they also lose the contralateral limb within 5 years following amputation and adversely affecting the quality of life. Early recognition and treatment of foot ulcers in diabetic patient is very critical for the successful outcome of the treatment. The conventional methods for the management of diabetic foot ulcer includes debridement of the wound, management of any infection, revascularization procedures when indicated, and off-loading of the ulcer. Other methods have also been used as add-on therapies, such as hyperbaric oxygen therapy, use of advanced wound care products (skin grafts, growth factors, matrix proteins), and negative-pressure wound therapy.

Some of the conventional wound healing techniques as present in the prior art are Gauze, wound gels, hydrocolloid dressings, alginates, hydrofiber dressings, foam dressings, Silver ions.

Gauze comes in a plethora of forms, sizes, shapes, and layered products. The most common gauze products are sterile or aseptic packaged individually, in packages of 2 or in bulk packages of 50 or 200 cotton gauze, woven, 8 or 12 layered, 4″×4″ or 2″×2″ sizes. Gauze may be used as both primary and secondary dressings and may come already impregnated with other substances such as calamine, petrolatum, wound gels, silver, etc. Also, combination products may have layers of gauze combined with layers of other wound products such as charcoal, alginates, adhesive backings, or borders. Care needs to be taken when using certain types of roll gauze or woven gauze as a primary dressing in a wound bed. Some gauze products may leave behind small pieces of organic cotton material, becoming foreign bodies in the wound and perhaps facilitating bacteria's growth or promoting hyper granulating tissue.

Wound gels come in amorphous gels (in tubes) or sheets of flexible semisolid gel. Wound gels are commonly made of organic polymers that maintain moisture in the wound bed and swell with water or wound drainage. Also, wound gel may contain silicone, water, glycerin, polyethylene oxide, alginate, or collagen. Typically the gel product is placed in the wound bed and covered with a secondary dressing to secure in place such as gauze or foam.

Hydrocolloid dressings are typically opaque, self-adherent “patch” type dressings made of sodium carboxy methyl cellulose, pectin, and gelatin mixed with polymers and adhesives. They also have a semipermeable film or foam sheet covering which makes them generally waterproof. However, waterproof does not mean it can be submerged in a pool. These dressings are flexible wafers of differing sizes, thicknesses, and shapes.

Alginates are super absorbent fibers typically composed of calcium alginate manufactured from brown seaweed that becomes gel-like when exposed to sodium-rich wound exudates. It resembles angle hair and is manufactured from brown seaweed. However, they are not recommended for dry or only slightly moist wound beds, as they will not remain a gel without the presence of moisture from the wound bed. Thus, they may dehydrate the wound bed or allow the wound bed to dry out. Also, some alginates may have silver incorporated into the fibers as an antimicrobial agent. Alginates typically require a secondary cover dressing such as gauze or ABD pad and are changed daily or as necessary to manage wound exudates. Hydrofiber dressings are non-wicking, absorptive primary dressings made of sodium carboxymethyl-cellulose fibers that absorb wound drainage and turn into a gel sheet. They may also keep the wound bed moist if the wound is sometimes dry. Hydrofibers act somewhat like alginate but will not promote hemostasis like alginates. Some hydrofiber dressings include 1.2% silver as an antimicrobial component. They are appropriate for full-thickness wounds with minimal to moderate amounts of drainage. They are typically changed once every 1 to 3 days and require a secondary cover dressing.

Foam dressings are typically both absorptive and protective. They may be selected to provide conforming padding and may be used in combination with other products such as alginates or hydrofibers if needed. Foams may be used as packing material in large wounds to fill dead space. Not all foam dressings are appropriate for infected wounds.

Silver ions may be incorporated in wound gels, woven fabric dressings, foam, rope, alginates, or hydrofiber dressings. Most silver fabric dressings are not very absorbent. They are used primarily to deliver silver ions to the wound bed for the silver's antimicrobial effects. However, silver alginates, hydrofibers, foams or composite dressings are absorbent. Silver ions are activated by wound exudates or water; some silver products should not be moistened with sodium chloride. Most ionic silver products should not be mixed with hydrogen peroxide or sodium hypochlorite solutions because the ions inactivate each other. Ionic silver products should not be combined with iodine products for the same reason. Silver dressings may need secondary dressings to hold them in place or provide extra absorption and may be changed daily up to every seven days, depending on the product.

Some of the conventional wound healing techniques as described in the prior art are Gauze, wound gels, hydrocolloid dressings, alginates, hydrofiber dressings, foam dressings, Silver ions.

There is no any disclosure of any such composition of cells having wound healing properties from the specific fraction of cultured peripheral blood mononuclear cells in a simple and cost-effective way. The present invention describes the composition and method to generate in vitro cells from the isolated peripheral blood mononuclear cells from the blood sample collected from a patient and preparing the therapeutic composition from the same. The said composition can be used for chronic non healing wounds.

In order to overcome the problems of longer duration of treatment and low patient compliance in these conventional wound healing techniques, the inventor of the present invention has found a novel therapeutic composition of cells isolated from peripheral blood mononuclear cells from the blood sample and a method to make an aqueous suspension for Injection, gel and patch from the same.

### Objective of the Invention

The main objective of the present invention is to provide novel therapeutic products generated from peripheral blood mononuclear cells in-vitro.

Another objective of the present invention is to provide a process of preparing novel therapeutic products generated from peripheral blood cells in vitro.

Another objective of the present invention is to provide novel therapeutic products an aqueous suspension for Injection, gel and patch generated from peripheral blood cells in vitro.

Another objective of the present invention is to provide novel therapeutic products an aqueous suspension for Injection, gel and patch which is simple and cost effective.

Another objective of the present invention is to provide novel therapeutic products an aqueous suspension for Injection, gel and patch which are having better patient compliance.

Another objective of the present invention is to provide a novel therapeutic products for treatment of wounds especially chronic non healing wounds.

### SUMMARY OF THE INVENTION

The main aspect of the present invention is to provide a novel therapeutic products generated from peripheral blood mononuclear cells in-vitro.

The other main aspect of the present invention is to provide a therapeutic product comprising gel composition which comprises Lympho-Myeloid Niches cell composition suspended in a biocompatible sterile collagen gel comprises 1-50% cell culture medium, 1-50% plasma or serum, 1-5% antibiotics and thickener, wherein the supplements cell culture medium, plasma or serum, antibiotics and thickener do not exceed 50% of the composition.

The other main aspect of the present invention is to provide a therapeutic product comprising patch composition which comprises Lympho-Myeloid Niches cell composition embedded in a patch comprises cell suspension medium containing 1-50% cell culture medium, 1-50% plasma or serum and 1-5% antibiotics wherein the volume of suspension medium can be kept as 0.1-2 ml for the 1 square centimetre of the patch and adding the cells suspension on to the collagen sheet by distributing uniformly.

The other main aspect of the present invention is to provide a therapeutic product comprising injection composition which comprises Lympho-Myeloid Niches cell composition supplied in a cell culture medium comprises 1-50% plasma or serum and 1-5% antibiotics in 1-10 ml volume in a prefilled syringe or vial.

Another aspect of the present invention is to provide a process of preparing therapeutic product comprising gel composition comprising the steps of,
a) recovering or isolating peripheral blood mononuclear cells (PBMC) from blood sample collected from a patient through ficoll gradient centrifugation;
b) resuspending the final cell pellet in RPMI medium containing 20% fetal bovine serum as growth medium;
c) counting and seeding PBMC in cell culture flask or vessel;
d) incubating the culture flask or vessel at 37° C. temperature in the presence of 5% CO₂;
e) removing non adherent cells from culture flask or vessel by gently shaking and followed by flushing the adherent cells with the RPMI medium;
f) maintaining the adherent cells in Lympho-Myeloid Niches (LMN), macrophages, lymphocytes, dendritic cell, endothelial cells, progenitor cells, and stem cells for long term by removing the old RPMI medium and then adding fresh RPMI medium once in every 2-3 days;
g) collecting the non-adherent cells generated and detached from the niche by centrifugation and harvesting the adherent cells from the culture using cell dissociating reagents or scraping the cells through cell scraper;
h) suspending the cells obtained from step e) in a biocompatible sterile collagen gel comprising 20% RPMI medium, 10% plasma or serum and 1% antibiotic and thickener wherein the supplements RPMI medium, plasma or serum, antibiotic and thickener should not exceed 50% of the composition, suspending the cells in the supplement and then mixing with the gel to make the final therapeutic product.

Another aspect of the present invention is to provide a process of preparing therapeutic product comprising patch composition comprising the steps of,
a) recovering or isolating peripheral blood mononuclear cells (PBMC) from blood sample collected from a patient through ficoll gradient centrifugation;
b) resuspending the final cell pellet in RPMI medium containing 20% fetal bovine serum as growth medium;
c) counting and seeding PBMC in cell culture flask or vessel;
d) incubating the culture flask or vessel at 37° C. temperature in the presence of 5% CO2;
e) removing non adherent cells from culture flask or vessel by gently shaking and followed by flushing the adherent cells with the RPMI medium;
f) maintaining the adherent cells in Lympho-Myeloid Niches (LMN), macrophages, lymphocytes, dendritic cell, endothelial cells, progenitor cells, and stem cells for long term by removing the old RPMI medium and then adding fresh RPMI medium once in every 2-3 days or longer;
g) collecting the non-adherent cells generated and detached from the niche by centrifugation and harvesting the adherent cells from the culture using cell dissociating reagents or scraping the cells through cell scraper;
h) suspending the cell composition from step e) in cell suspension medium containing sterile 20% RPMI medium supplemented with 10% plasma or serum and 1% antibiotic, the volume of suspension medium can be kept as 0.1-2 ml for the 1 square centimetre of the collagen sheet, using the biocompatible sterile collagen sheet and adding the cells suspension on to the collagen sheet by distributing uniformly.

Another aspect of the present invention is to provide a process of preparing therapeutic product comprising injection composition comprising the steps of,
a) recovering or isolating peripheral blood mononuclear cells (PBMC) from blood sample collected from a patient through ficoll gradient centrifugation;
b) resuspending the final cell pellet in RPMI medium containing 20% fetal bovine serum as growth medium;
c) counting and seeding PBMC in cell culture flask or vessel;
d) incubating the culture flask or vessel at 370 C temperature in the presence of 5% CO2;
e) removing non adherent cells from culture flask or vessel by gently shaking and followed by flushing the adherent cells with the RPMI medium;
f) maintaining the adherent cells in Lympho-Myeloid Niches (LMN), macrophages, lymphocytes, dendritic cell, endothelial cells, progenitor cells, and stem cells for long term by removing the old RPMI medium and then adding fresh RPMI medium once in every 2-3 days or longer;
g) collecting the non-adherent cells generated and detached from the niche by centrifugation and harvesting the adherent cells from the culture using cell dissociating reagents or scraping the cells through cell scraper;
h) suspending the cellular composition from step e) in a sterile normal saline comprises 10% plasma or serum and 1-5% antibiotics in 1-10 ml volume in a prefilled syringe or vial and closing airtight with rubber stopper and crimping.

### BRIEF DESCRIPTION OF DRAWINGS

FIG. 1: The identified novel Lympho-Myeloid Niches (LMN) with characteristics of giving rise to myeloid and lymphoid cells in the cultures of peripheral blood cells and further characterized their regenerative potential to generate large number of cells in vitro and cellular composition of colonies—mainly T cells and macrophages.

FIG. 2: The identified novel Lympho-Myeloid Niches (LMN) with characteristics of giving rise to myeloid and lymphoid cells in the cultures of peripheral blood cells and further morphologies of proliferating cell colonies and cells in scratch assay.

FIG. 3: BrDU labelling experiments showed the new cell generation potential of the LMN. BrDU labelling confirmed that the cells in the Niche are highly proliferative and expanding in nature.

FIG. 4: Methodology of gel preparation.

FIG. 5: Viability of the cells in collagen gel and collagen gel mixed with plasma or serum imaged under fluorescent microscope following day 2 of live cell staining with CFSE (green) and DiL (red).

FIG. 6: Methodology and cells embedded on to collagen sheet and stained with Giemsa stain. Top: shows the diagrammatic representation of the patch formulation. Bottom: The cell embedded patch stained with Giemsa stain.

FIG. 7: The cells in the collagen patch were fixed in Methanol and stained with Giemsa stain and imaged under a microscope.

FIG. 8: Characterization of the blood cells prior to in vitro culture for developing therapeutic product. Dot blot analysis of the flow cytometry data shows the presence of a large proportion of T cell in the starting peripheral blood mononuclear cell population. Top row: Isotype control tube; Bottom row: Specific antibody stained tube.

FIG. 9: Characterization of the peripheral blood cells prior to in vitro process for developing therapeutic product. Dot blot analysis of the flow cytometry data shows the presence of a very small fraction of CD31, CD10, CD34 positive cells in the blood. Top row: Isotype control tube; Bottom row: Specific antibody stained tube.

FIG. 10: Characterization of the therapeutic product for the expression of CD31 antigens on the surface following in vitro process. Dot blot and histogram analysis of the flow cytometry data shows the presence of a specific population of cells CD31 antigens.

FIG. 11: Characterization of the therapeutic product for the expression of CD10 antigens on the surface following in vitro process. Dot blot and histogram analysis of the flow cytometry data shows the presence of a specific population of cells CD10 antigens.

FIG. 12: Characterization of the therapeutic product for the expression of CD34 antigens on the surface following in vitro process. Dot blot and histogram analysis of the flow cytometry data shows the presence of a specific population of cells CD34 antigens.

FIG. 13: Characterization of the cells of therapeutic product for the expression of CD10 antigen by fluorescent microscopy. The cells were stained with mouse anti-human CD10 primary antibody and then stained with anti-mouse antibody labelled with Alexa-488 fluorochrome. Top row: cells stained without CD10 antibody. A—Phase contrast image, B—fluorescent image. Bottom row: Cells stained with CD10 antibody. C— Phase contrast image, D—fluorescent image.

FIG. 14: Characterization of the migration property of the therapeutic product following a scratch in the culture surface. The scratch area was observed under an inverted microscope (brightfield/phase contrast) and imaged. A—100×, B—200×, C—400× Magnification.

FIG. 15: Therapeutic product derived from chicken using the same protocol used for generating human product, which showed similar phenotypic properties. Images A and C: 100× magnification; B and D: 200× magnification. Images AB and CD were taken different days of in vitro process.

FIG. 16: Therapeutic product derived from in vitro processing of Chicken cells showed migratory potential similar to the human cells. Images were taken at different magnifications. A: 100×, B: 200×. C: 400×.

FIG. 17: Representative images showing the healing of wound observed on different days following the treatment. The excision wound was created using a sterile scalpel and the wound size was measured prior treatment and on different interval following the treatment. In each bird, two wounds were created and one was treated and another was kept as untreated wound.

FIG. 18: The photograph of excision wound taken a) on Day 1 (just prior to initiation of treatment) and b) On 11^{th} day. Percent change in wound area was calculated for each animal and average change is reported.

FIG. 19: The representative histological sections of skin from each group.

FIG. 20: The image of wound of a 55 year female suffering from Elephantiasis, having a nonhealing wound for more than 2 months. The images are shown before the treatment (A) and 15 days after the treatment (B).

FIG. 21: The image of a wound of a 65 year old male, suffering from diabetic and atherosclerotic, had non healing wound after amputation of lower limb for more than 2 months. The images are shown before the treatment (A) and 30 days after the treatment (B).

### DESCRIPTION OF THE INVENTION

The present invention relates to novel therapeutic products generated from peripheral blood cells in vitro and process of preparing the novel therapeutic products.

As used herein, the term “formulation” or “composition” unless otherwise defined refers to injection and/or patch, gel for topical and injectable pharmaceutical dosage forms or suspension of the invention.

The term “therapeutic product” used herein are products having a beneficial effect on the body or mind and producing a useful or favourable result or effect relating to the treatment of disease or disorders.

As per one embodiment, the term “biocompatible” used herein is a term describing the property of a material being compatible with living tissue. Biocompatible materials do not produce a toxic or immunological response when exposed to the body or bodily fluids.

As per one embodiment, the term “plasma or serum” used herein is a plasma with high concentration of platelets, which contains a large amount of proteins which enhance body's natural healing response. Serum is the liquid that remains after the blood has clotted and consists of 90% water with dissolved hormones, proteins, minerals, and carbon dioxide.

As per one embodiment, the term “cell culture medium” used herein is a growth medium used in cell culture which support the growth of microorganisms, cells, or small plants and having an appropriate source of energy and compounds which regulate the cell cycle.

As per another embodiment, the present invention provides a novel method of formulating an aqueous suspension for Injection, Gel, and Patch.

As per one embodiment, the present invention contains in vitro novel Lympho-Myeloid Niches (LMN) in large number of cells, in the cultures of peripheral blood cells obtained from the human blood. The in vitro cells generated from these niches have wound healing properties especially for chronic non healing wounds.

As per one of the preferred embodiments, the present invention provides a therapeutic product comprising gel composition which comprises Lympho-Myeloid Niches cell composition suspended in a biocompatible sterile collagen gel comprises 1-50% cell culture medium, 1-50% plasma or serum, 1-5% antibiotics and thickener, wherein the supplements cell culture medium, plasma or serum, antibiotics and thickener do not exceed 50% of the composition.

As per one of the preferred embodiments, the present invention provides a therapeutic product comprising patch composition which comprises Lympho-Myeloid Niches cell composition embedded in a patch comprises cell suspension medium containing 1-50% cell culture medium, 1-50% plasma or serum and 1-5% antibiotics wherein the volume of suspension medium can be kept as 0.1-2 ml for the 1 square centimetre of the patch and adding the cells suspension on to the collagen sheet by distributing uniformly.

As per one of the preferred embodiments, the present invention provides a therapeutic product comprising injection composition which comprises Lympho-Myeloid Niches cell composition supplied in a cell culture medium comprises 1-50% plasma or serum and 1-5% antibiotics in 1-10 ml volume in a prefilled syringe or vial.

As per one embodiment of the present invention, the in vitro generated Lympho-Myeloid Nich cell composition comprises macrophages, lymphocytes, dendritic cell, endothelial cells, progenitor cells, and stem cells.

As per one embodiment of the present invention, the cell culture medium used for growing the cells are selected from MEM, DMEM, F12, DMEM/F12, IMDM, M-199, RPMI medium, serum free medium, T cell medium, stem cell medium, keratinocyte culture medium, cell specific culture medium, normal saline, ringer lactate solution, phosphate buffered solution, balanced salt solution and combination thereof.

As per one embodiment of the present invention, the plasma or serum used for preparing the cell based therapeutic products may be selected from the human or animal origin, autologous or allogeneic, purified serum or plasma components. In the present invention, autologous plasma is a most preferred plasma.

As per one embodiment of the present invention the antibiotic used for preparing the cell based therapeutic product may be selected from the group consisting the group consisting gentamicin, kanamycin, penicillin, streptomycin, doxycycline, tetracycline, ciprofloxacin, amoxicillin, cefuroxime and cefepime. In a most preferred embodiment, Penicillin, streptomycin, gentamicin are used as an antibiotic.

As per one embodiment of the present invention, the thickener used for preparing the cell based therapeutic gel and patch product may be selected from the group consisting of carbohydrate, poly olefinic, pyrrolidone, silicone, and combination thereof.

As per one embodiment of the present invention, the patch used for embedding cells to the cell based therapeutic patch product may be selected from collagen, chitosan, biocompatible membrane, biocompatible scaffold, biocompatible polymers, extracellular matrix, bioabsorbable materials, hydrogels, methylcellulose, and combination thereof.

As per one embodiment, the novel therapeutic product of the present invention is an autologous combination of in vitro generated cell based therapeutic product. Its composition includes different cells including but not limited to cells of lympho-myeloid niches, stem cells, progenitor cells, endothelial cells and macrophages which are generated in vitro from blood mononuclear cells. It is formulated either in the form of aqueous suspension for injection or a gel or a patch.

Another main embodiment of the present invention provides a novel method of preparing the novel therapeutic products of the present invention, an aqueous suspension for injection, a gel and a patch.

As per one embodiment of the present invention the method for formulating an aqueous suspension using blood collected from a patient for gel composition comprises the steps of,
a) recovering or isolating peripheral blood mononuclear cells (PBMC) from blood sample collected from a patient through ficoll gradient centrifugation;
b) resuspending the final cell pellet in RPMI medium containing 20% fetal bovine serum as growth medium;
c) counting and seeding PBMC in cell culture flask or vessel;
d) incubating the culture flask or vessel at 37° C. temperature in the presence of 5% CO₂;
e) removing non adherent cells from culture flask or vessel by gently shaking and followed by flushing the adherent cells with the RPMI medium;
f) maintaining the adherent cells in Lympho-Myeloid Niches (LMN), macrophages, lymphocytes, dendritic cell, endothelial cells, progenitor
g) cells, and stem cells for long term by removing the old RPMI medium and then adding fresh RPMI medium once in every 2-3 days;
h) collecting the non-adherent cells generated and detached from the niche by centrifugation and harvesting the adherent cells from the culture using cell dissociating reagents or scraping the cells through cell scraper;
i) suspending the cells obtained from step e) in a biocompatible sterile collagen gel comprising 20% RPMI medium, 10% plasma or serum and 1% antibiotic and thickener wherein the supplements RPMI medium, plasma or serum, antibiotic and thickener should not exceed 50% of the composition, suspending the cells in the supplement and then mixing with the gel to make the final therapeutic product.

As per another embodiment of the present invention, filling the therapeutic product in final formulation in a sterile tube or a syringe under sterile aseptic condition and storing it at 4-10° C. until the use in patients. The product should be used within 24 hours from the dispatch from the manufacturing laboratory. Using the product for local topical application over the wound following cleaning and debridement and applying the dressing to retain the gel on the wound.

As per one embodiment of the present invention the method for formulating an aqueous suspension using blood collected from a patient for patch composition comprises the steps of,
a) recovering or isolating peripheral blood mononuclear cells (PBMC) from blood sample collected from a patient through ficoll gradient centrifugation;
b) resuspending the final cell pellet in RPMI medium containing 20% fetal bovine serum as growth medium;
c) counting and seeding PBMC in cell culture flask or vessel;
d) incubating the culture flask or vessel at 37° C. temperature in the presence of 5% CO2;
e) removing non adherent cells from culture flask or vessel by gently shaking and followed by flushing the adherent cells with the RPMI medium;
f) maintaining the adherent cells in Lympho-Myeloid Niches (LMN), macrophages, lymphocytes, dendritic cell, endothelial cells, progenitor cells, and stem cells for long term by removing the old RPMI medium and then adding fresh RPMI medium once in every 2-3 days or longer;
g) collecting the non-adherent cells generated and detached from the niche by centrifugation and harvesting the adherent cells from the culture using cell dissociating reagents or scraping the cells through cell scraper;
h) suspending the cell composition from step e) in cell suspension medium containing sterile 20% RPMI medium supplemented with 10% plasma or serum and 1% antibiotic, the volume of suspension medium can be kept as 0.1-2 ml for the 1 square centimetre of the collagen sheet, using the biocompatible sterile collagen sheet and adding the cells suspension on to the collagen sheet by distributing uniformly.

As per one embodiment, final therapeutic product patch is keeping in a sterile plastic bag or sealing under sterile aseptic condition and storing it at 4-10° C. until the use in patients. The product should be used within 24 hours following the dispatch from the manufacturing laboratory. Using the product for local topical application over the wound following cleaning and debridement and applying the dressing to retain the patch on the wound.

As per one preferred embodiment of the present invention the method for formulating an aqueous suspension using blood collected from a patient for injection comprises the steps of,
a) recovering or isolating peripheral blood mononuclear cells (PBMC) from blood sample collected from a patient through ficoll gradient centrifugation;
b) resuspending the final cell pellet in RPMI medium containing 20% fetal bovine serum as growth medium;
c) counting and seeding PBMC in cell culture flask or vessel;
d) incubating the culture flask or vessel at 370 C temperature in the presence of 5% CO₂;
e) removing non adherent cells from culture flask or vessel by gently shaking and followed by flushing the adherent cells with the RPMI medium;
f) maintaining the adherent cells in Lympho-Myeloid Niches (LMN), macrophages, lymphocytes, dendritic cell, endothelial cells, progenitor cells, and stem cells for long term by removing the old RPMI medium and then adding fresh RPMI medium once in every 2-3 days or longer;
g) collecting the non-adherent cells generated and detached from the niche by centrifugation and harvesting the adherent cells from the culture using cell dissociating reagents or scraping the cells through cell scraper;
h) suspending the cellular composition from step e) in a sterile normal saline comprises 10% plasma or serum and 1-5% antibiotics in 1-10 ml volume in a prefilled syringe or vial and closing airtight with rubber stopper and crimping.

As per another embodiment of the present invention, storing of the vials for injection is done by closing airtight with rubber stopper and crimping, keeping it at 4-10° C. and sending it to the clinic for the use in patient. It should be used in the patient within 24 hours from the time of dispatch from the manufacturing laboratory.

As per another embodiment of the present invention, administering the aqueous suspension of injection by taking out the composition in a Tuberculin syringe fitted with 31 G needle and injecting it in to the skin adjacent to surrounding the wound at a depth of 2-3 mm and multiple injections spread out evenly covering the entire wound.

As per another embodiment of the present invention, the cell viability of the in vitro generated cells can be determined by trypan blue staining and using the cells for therapeutic purposes. As an optional step, staining the cells for markers such as CD10, CD34, CD31, CD3, CD4, CD33 and CD14 and analysing in a flow cytometer for understanding the cellular composition.

As per another embodiment of the present invention, cell dissociating reagents used for collecting adherent cells from the LMN are selected from trypsin, trypsin-EDTA, collagenase, elastase, accutase, dispase and EDTA solutions.

As per one embodiment the therapeutic products of the present invention are aqueous suspension for injection, a gel and a dermal patch which are used for the treatment of wounds preferably chronic non healing wounds.

As per another embodiment the therapeutic product of the present invention provides gel wherein the cellular composition is suspended in the gel containing extracellular matrix, which is used for local topical application over the wound following debridement.

As per another embodiment the therapeutic product of the present invention provides Patch, a dermal patch wherein the cellular composition is embedded in a collagen patch which can be applied over the wound directly following cleaning and debridement.

As per another embodiment the therapeutic product of the present invention provides aqueous suspension for Injection wherein the cellular composition is supplied in liquid suspension formulation in 1-2 ml volume containing specific number of cells as an injection for administering in to the wound following proper debridement. It is to be taken out in a Tuberculin syringe fitted with 27 G or 31 G needle and injected in to the wound at a depth of 2-3 mm at multiple sites spread out evenly covering the entire wound. Then the wound can be dressed properly with aseptically using clean gauze.

As per one embodiment the therapeutic products of the present invention are used for the treatment of chronic non healing wounds which is selected from the group consisting of a the group consisting of a chronic wound, non-healing surgical wound, diabetic foot ulcers, burn, an infected tissue or wound, vascular ulcers, arterial ulcers, infarction, necrosis, gangrene, and bed sore.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### Example 1: Preparation of Cell Based Composition

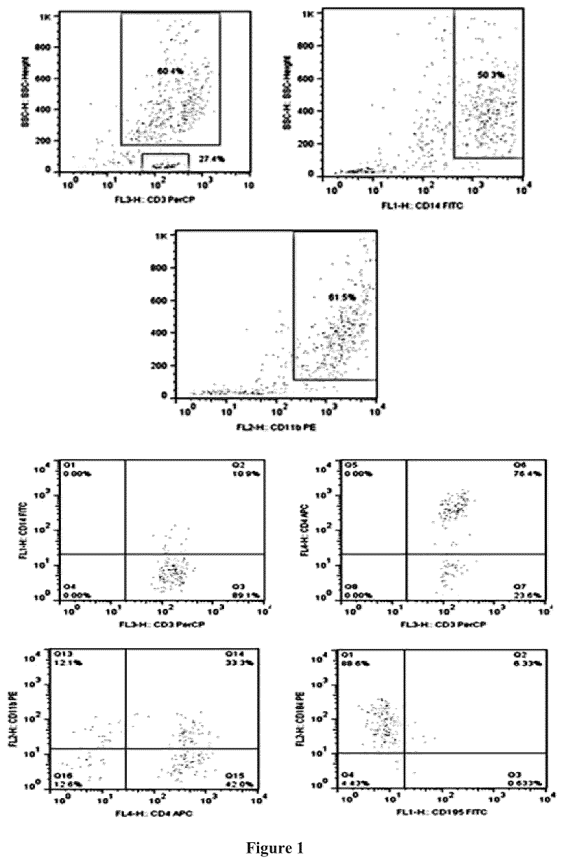

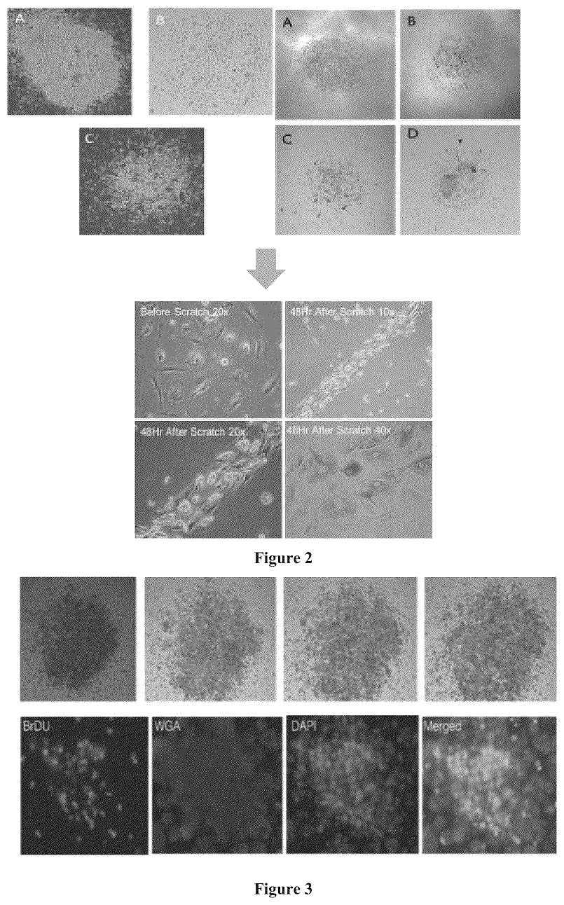

For generating the cellular components of proposed therapeutic product, the PBMC was obtained from the blood using ficoll gradient centrifugation. Briefly, the blood was diluted and overlaid on the ficoll solution. PBMC was cultured using RPMI-1640 medium supplemented with 20% fetal bovine serum and 10% human serum or plasma. Human plasma or serum can be optional for short term cultures. Lympho-myeloid niches formation can be seen even in the absence of human autologous plasma. Optimization of the cell concentration revealed that 25000-50000 cells were needed to get 1-2 niches in single well of 48 well plate in 10-20% of the wells. The cells of these niches were characterized by flow cytometry and it was found that they are primarily myeloid and lymphoid cells as represented in FIG. 1. For flow cytometry, harvested cells were centrifuged, suspended in RPMI-20%, counted and 50 μl aliquots were incubated with fluorochrome-labeled antibodies for 20 minutes at room temperature in the dark. Three ml of DPBS containing 1% FBS (wash buffer) was added and tubes were briefly vortexed, followed by centrifugation to pellet the cells. Cells were suspended in 1% paraformaldehyde and acquired on a Flow cytometer (BD Biosciences) and analysed using FlowJo Software. Ten-to-forty thousand events were acquired per tube depending on the frequency of populations of interest. Live cell imaging and BrDU labelling experiments showed the new cell generation potential of the LMN as represented in FIG. 2. Bromodeoxyuridine (BrdU) is a thymidine analog which is incorporated into the newly synthesised DNA during DNA replication. For BrDU labelling the cells were grown in 24-well plates as described above. BrDU was added at a final concentration of 50 mM at days 5, 7 or 10 of culture. Cells were incubated with BrdU for 18-48 hours. Fixation was performed using 70% ethanol for 30 minutes, followed by a PBS wash, incubation with 0.5% Triton X-100 in PBS for 10 minutes to permeabilize the cells and then a 1 hour incubation with 2N HCl to denature the DNA. The wells were then washed twice using PBS pH 8.0 and incubated for 2 hours with FITC-conjugated mouse anti-BrdU (BD Biosciences) diluted 1:4, in DPBS supplemented with 0.5% Tween 20. Following 3 washes in PBS, the cells were counterstained by incubation with 2.8 mM DAP1 (Molecular Probes) for 10 minutes, washed and examined using fluorescent microscope.

### Example 2: Formulation of Aqueous Suspension for INJECTION

For preparing the in the form of injection, the cell composition was suspended in sterile normal saline containing 10% autologous plasma in 2 ml volume in a 2 ml vial and closed airtight with rubber stopper and crimp. It was kept in an ice pack at 4-8° C. and sent to the clinic for the use in patient. It should be used in the patient within 24 hours from the time of manufacturing. The product will be taken out in a Tuberculin syringe fitted with 31 G needle and injected in to the wound at a depth of 2-3 mm as multiple injections spread out evenly covering the entire wound.

### Example 3: Formulation of Gel

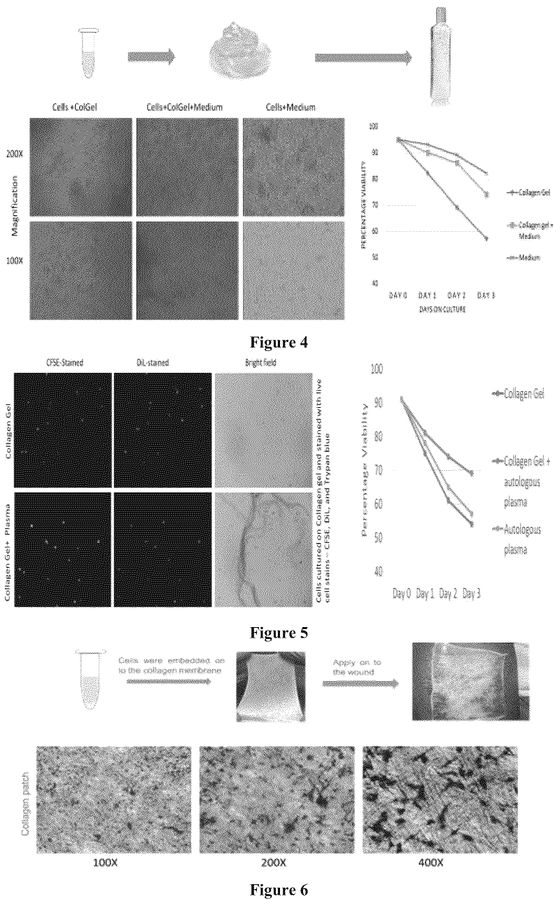

For preparing the gel, the cell composition were suspended in a biocompatible collagen gel. The viability of cells in the gel and gel supplemented with RPMI medium and autologous plasma were tested. The cells were cultured in different conditions and viability was determined by trypan blue staining. In some experiments, the cells were also stained with live cell stains such as CFSE and DIL (ThermoFisher, USA) and seeded on to a 24 well plate at a concentration of 1 million in 2 ml volume. 0.5 ml was removed each day and pelleted and then counted for percentage of viability by trypan blue staining. The cells were also imaged under a fluorescent microscope for determining the presence of viable fluorescent cells. It was found that the cells supplemented with either autologous plasma or RPMI medium has better viability (FIGS. 3 and 4). The final formulation of the gel was supplemented with the collagen gel with 20% medium and 10% plasma to achieve better viability of cells. It will be used for local topical application over the wound following debridement. This will fill the space and provide environment for cells to grow and promote healing.

### Example 4: Formulation of Patch

For preparing Patch, the cell composition were embedded on to a biocompatible collagen sheet which can be applied over the wound directly following debridement. The cultured cells suspended in the culture medium were added to the collagen sheet and cultured for 3 days. The cell embedded sheet was removed and washed with DPBS thrice and then fixed with methanol. The sheet was stained with Giemsa stain and imaged under a microscope as represented in FIG. 5. To understand the long-term viability of cells embedded onto the collagen sheet, cells were stained with live cell stain DiL and then embedded onto the collagen sheet. On day 3, the sheet was washed with thrice with DPBS and imaged under the fluorescent microscope for the presence of viable cells. The sheet was fixed, stained with Giemsa and imaged under a microscope as represented in FIG. 6.

### Example 5: In Vitro Validation of Therapeutic Product

Studies were carried out studies for validation of the therapeutic product for promoting wound healing. In vitro studies were carried out to understand the expression of CD10 marker and migration potential. The expression of CD10 antigen was studied on the cell product by immunostaining and then analysing using flow cytometer and fluorescent microscope. CD10 is a 90-110-kDa cell surface zinc-dependent metalloprotease that is widely expressed on progenitor cells and it is considered as one of the markers for stem/progenitor cells and its expression in specific cell indicates the regeneration potential and thereby promoting the healing of wound.

### Example 6: Analysis of Expression of Cd 10 by Flow Cytometry

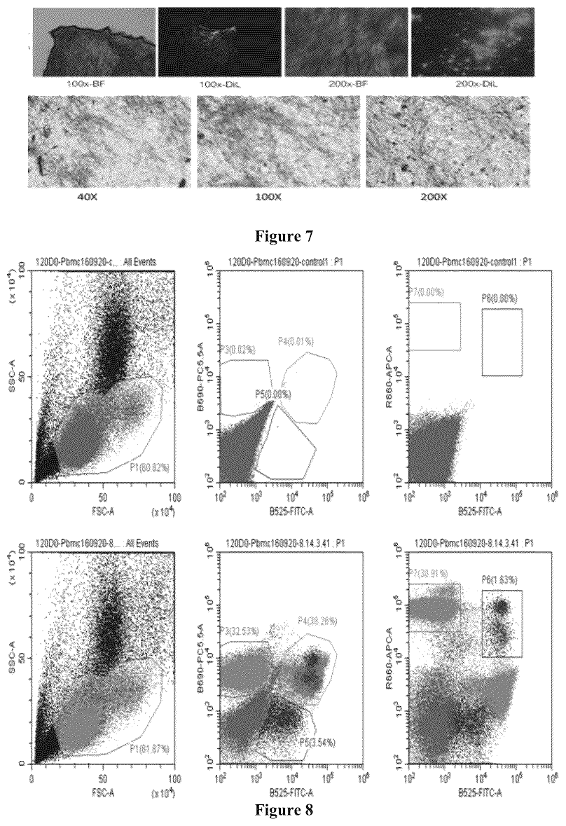

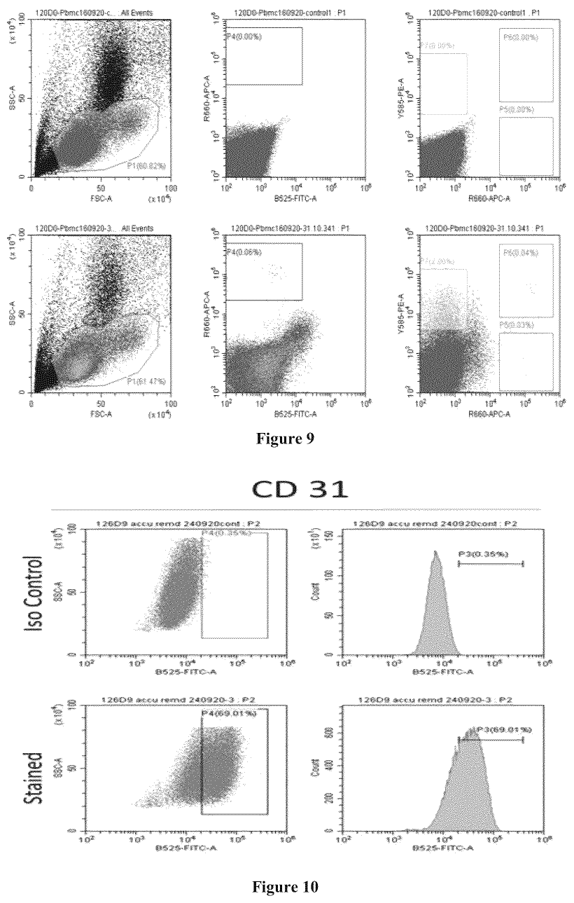

For studying the expression of CD10, the cells were harvested and stained with anti-CD10 antibody labelled with PE flurochrome. Along with anti-CD10 antibody, the anti-CD31 and anti-CD34 antibodies were also added. The CD31 is a marker for endothelial cells as it is widely expressed on endothelial cells, which have potential to form new blood vessel in the body. CD34 is a marker for hematopoietic stem cell. The CD31 antibody labelled with FITC flurochrome and the CD34 antibody labelled with APC flurochrome was used in this study. For understanding the expression of these important antigens, the peripheral blood mononuclear cells were obtained from normal donor blood and stained with these antibodies to understand the presence of these cells present in blood. In addition to these markers, markers for T cells were also included. Thus, the cells prior to any procedure, and the cells obtained after in vitro process were studied to understand the presence of cells expressing these antigenic markers. For immunostaining, two 5 ml falcon tubes with snap cap were taken, one tube was stained with CD31-FITC, CD10-PE, and CD34-APC antibodies whereas the other tube was stained with isotype control antibodies. In each tube, 0.5 million cells were taken, and the three antibodies were added and incubated for 20 minutes in dark. The tubes were mixed well once in every 10 minutes. Following incubation, 3 ml of phosphate buffered saline with 1% foetal bovine serum (wash buffer) was added and mixed well by vortex. The tubes were centrifuged at 2000 rpm for 5 minutes in a centrifuge to pellet the cells. Then the buffer from each tube was discarded by inverting the tube, then 3 ml of wash buffer was added again to the cell pellet and mixed by vortex, and centrifuged again as mentioned earlier. After discarding the wash buffer, 300 microlitre of 1% paraformaldehyde was added and mixed by vertexing. The cells were acquired and analysed in a flow cytometer. Hundred thousand cells were acquired in from each tube and the data was analysed for the presence of cells having CD10, CD31, and CD34 antigens in the surface. The data is represented in FIGS. 7 and 8. It shows the presence of cells with specific marker of interest in the starting blood cell population and in the harvested therapeutic cell therapy product. FIG. 7 shows the presence of some normal blood cells such as T cell expressing CD3, CD4, and CD8 markers and the FIG. 8 shows the presence of cells expressing CD10, CD31 and CD34 antigen in the starting population prior to culture. FIG. 9 shows the presence of these cells in the therapeutic product obtained following in vitro culture process.

### Example 7: Analysis of Expression of Cd10 by Microscopy

For studying the expression of CD10 on the therapeutic product, the cells were fixed using 4% paraformaldehyde and stained with mouse anti-human CD10 primary antibody followed by secondary goat anti-mouse antibody labelled with Alexa-488 flurochrome. The cells were washed thrice and observed under fluorescent microscope (Olympus, Japan) and imaged. Cells stained with same procedure except the addition of primary antibody was used as negative control. A proportion of stained cells showed the expression of CD10 antigen on their surface as represented in FIG. 10.

### Example 8: Analysis of Migration Potential

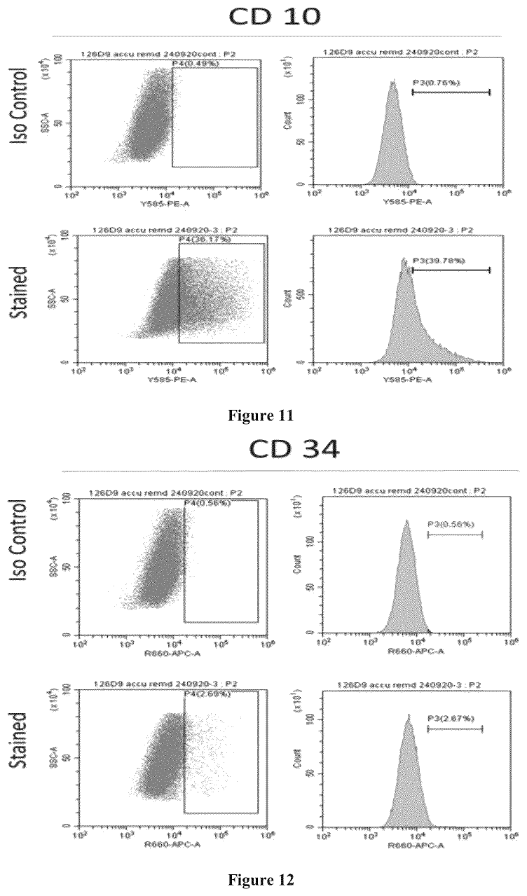

The therapeutic cells were also validated for their migration potential which is critical for promoting wound healing. For this, scratches were made on the plastic surface and the migration potential the product was observed under an inverted microscope for 5 days. The FIG. 11 shows that the cells can migrate to the scratch area and concentrate over there to promote wound healing.

### Example 9: In Vivo Validation of the Efficacy of THERAPEUTIC PRODUCT IN CHICKEN MODEL

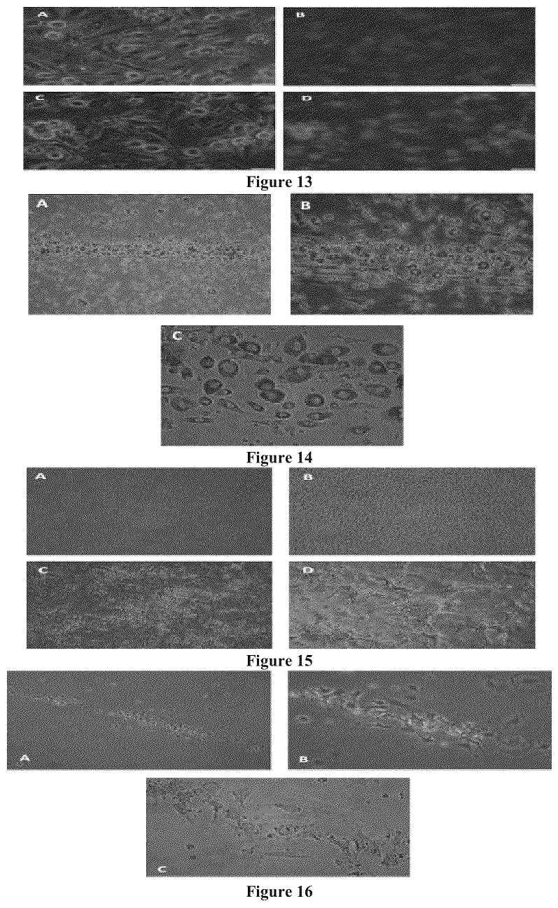

A novel wound healing model in Chicken was developed for studying the therapeutic potential of the product to promote wound healing in an autologous manner. Same protocol optimized for developing therapeutic product from human was followed to develop therapeutic product in Chicken as represented in FIG. 12. The cell product was studied for the migration potential in scratch assay, which confirmed that they have migratory potential as represented in FIG. 13. A novel excision wound model in chicken was developed and created an excision wound in the skin of chicken and injected the therapeutic product and studied their wound healing potential. For this experiment the therapeutic product was derived following the same protocol used for human cells. Wound was created on day 0 by removing a piece of 400 mm² skin tissue using a sterile scalpel on both side of the body under the wings. The therapeutic product was applied into the wound. In each bird, the wound on the left side of the body was treated using injectable product on four sides of the wound subcutaneously around the wound. The wound on right side of the body was kept as untreated control. The wounds were followed up for 10 days and images were taken at different interval. Representative images of the treated and untreated wound were given in the FIG. 14. The area of wound was measured by measuring the length and width of the wound at the longest position in millimeter (mm) and the wound area was estimated by multiplying these width and length and expressed in square (Sq) mm.

TABLE 1
Measurement of the wound area on before (day 0) and after (day 10) treatment.

| | DAY-0 | | DAY-10 | | % of Wound | |
|---|---|---|---|---|---|---|
| | (Wound area in Sq mm) | | (Wound area in Sq mm) | | Reduction | |
| Animal No | Untreated | Treated | Untreated | Treated | Untreated | Treated |
| AN1002 | 378 | 418 | 135 | 35 | 64 | 92 |
| AN1006 | 380 | 462 | 224 | 54 | 41 | 88 |
| AN1015 | 437 | 552 | 153 | 108 | 65 | 80 |
| AN1013 | 360 | 420 | 154 | 45 | 57 | 89 |

### Example 10: In Vivo Validation of the Efficacy of the THERAPEUTIC PRODUCT IN MICE MODEL

Therapeutic products were prepared in three different formulations injectable, gel and dermal patch using the in vitro generated cells of interest. The therapeutic products in different formulations were tested in mice using the excision wound model. The method and result of the mice model study are given below. The study was conducted to determine the efficacy of different cell-based formulations for their wound healing efficacy in excision wounds in mice. The study was carried out on limited number of animals to compare the efficacy of different formulations. Product details:
a) Formulation-I: an aqueous suspension for injectable formulation to be administered on the wound through superficial (1-2 mm depth) injections.
b) Formulation-II: a gel formulation to be applied on the wound externally.
c) Formulation-III: a dermal patch to be applied on the wound externally

Fifteen mice which met the study requirements were randomly selected from the animal housing facility and placed in individual cages. They were grouped in to five equal groups as described below in Table-2:

TABLE 2
Study Groups and Treatment allocation

| | Animal Species | No. of | | Treatment |
|---|---|---|---|---|
| Groups | and Strain | Animals | Treatment | Period |
| Group-I | Swiss Albino | 3 | Saline | 10 days |
| | Mice | | | |
| Group-II | Swiss Albino | 3 | Formulation-I (Inj) | 10 days |
| | Mice | | | |
| Group-III | Swiss Albino | 3 | Formulation-II (Gel) | 10 days |
| | Mice | | | |
| Group-IV | Swiss Albino | 3 | Formulation-III | 10 days |
| | Mice | | (Patch) | |
| Group-V | Swiss Albino | 3 | Formulation-I + II + III | 10 days |
| | Mice | | | |

The animal's dorsal skin was shaved off and 24 hours later were anaesthetized using ketamine (40 mg/Kg, i.p.). Approximately 200 sq·mm sized skin was excised out. The entire skin up to muscle layer was excised and the wound area was cleaned with sterile saline using cotton. The photograph of the wound was taken. Once the oozing of the blood stopped i.e. approximately 30 min after the wound creation, the respective drug treatments were applied and covered with plastic sheet and then secured using adhesive tape. The drug application was applied once only, and the animals were maintained in individual cages for ten days. Every day the animals were monitored for their general health. On day 11 the animals were euthanized by exposure to excess of ether. The wound area was photographed and skin area just adjacent to the wound was taken out for histology study. These excised skins were sectioned using microtome. The 10 μm sections were stained using hematoxylin eosin stain and observed under microscope (100× and 450× magnification) to determine the quality of healing. The wound photographs were used to determine the wound area using ImageJ software. The % reduction in wound area from the day of creating wound to that on 11^{th} day was considered as the wound healing efficacy of the treatment. The treatment groups included three groups for three different formulation and fourth group in which all the three formulations were applied at a time.

Wound area: The wound area was determined on day 1 and on day 11 from the photographs of the wound using ImageJ software. The representative photographs of a control animal taken on day 1 and 11 are shown in FIG. 15 below. The wound area prior to treatment on day 1, and on day 11 following treatment were compared to determine the percentage of wound size reduction and it is given in Table 3.

TABLE 3
Effect of Different Formulations on wound Area

| | | | % Reduction |
|---|---|---|---|
| | Wound Area (Sq.mm) | | in Wound |
| Group | Day1 | Day11 | Area |
| Saline (Control) | 119.030 | 42.915 | 58.36 |
| Formulation-I | 168.750 | 54.440 | 67.74 |
| (Injection) | | | |
| Formulation-II (Gel) | 131.641 | 33.011 | 75.55 |
| Formulation-III (Patch) | 145.286 | 52.099 | 64.14 |
| Formulation-I + II + III | 198.445 | 74.205 | 62.61 |

Histology of skin adjacent to wound area: The skin adjacent to wound area was excised out and used for histological sectioning. This skin is thus from recently healed area and thus can help in understanding the healing process. The dense deposition of collagen fibers indicative of fibrous deposits as a part of natural healing. The representative sections, one from each group, is presented in the FIG. 16. Group-I: Photomicrograph of excised skin obtained from control animals. (c) Collagen fibers in dermis layer, (e) elastin fibers in dermis layer. (a) Adipose tissue, (epi) epidermis. Group-II: Photomicrograph of excised skin obtained from animals that received Injection formulation. The presence of sebaceous glands (s), hair follicles (h) less dense collagen fibers (c) can be seen. Group-III: Photomicrograph of excised skin obtained from animals that received Gel Formulation. The presence of hair follicles (h), Collagen (c). Group-IV: Photomicrograph of excised skin obtained from animals that received Patch formulation. Dense collagen fibers (c), presence of hair follicles (h). Healing observed. Group-V: Photomicrograph of excised skin obtained from animals that received all the three formulations. The good number of capillaries (cap) and less dense collagen (c) suggest good quality of healing.

### Example 11: Clinical Study of the Therapeutic Gel Product

The therapeutic product safety and efficacy was studied in patients with chronic nonhealing wounds. The product formulated in the form of gel was applied on to the wounds of patients with chronic nonhealing wound. Prior to the application, the wound was cleaned and rinsed with normal saline. Then the therapeutic product in the form of gel was applied directly onto the wound. The wound was covered with transparent dressing material such as Tegaderm. Gauze dressing was done over the transparent dressing. The wound was inspected on day 5 following application for presence of any infection. If there is no infection, the transparent dressing was not removed. On day 10, the transparent dressing was removed and the wound was washed using normal saline. Then sterile gauze with saline was applied on to the wound and gauze dressing was done. The wound was inspected once in every five days till day 30 following application.

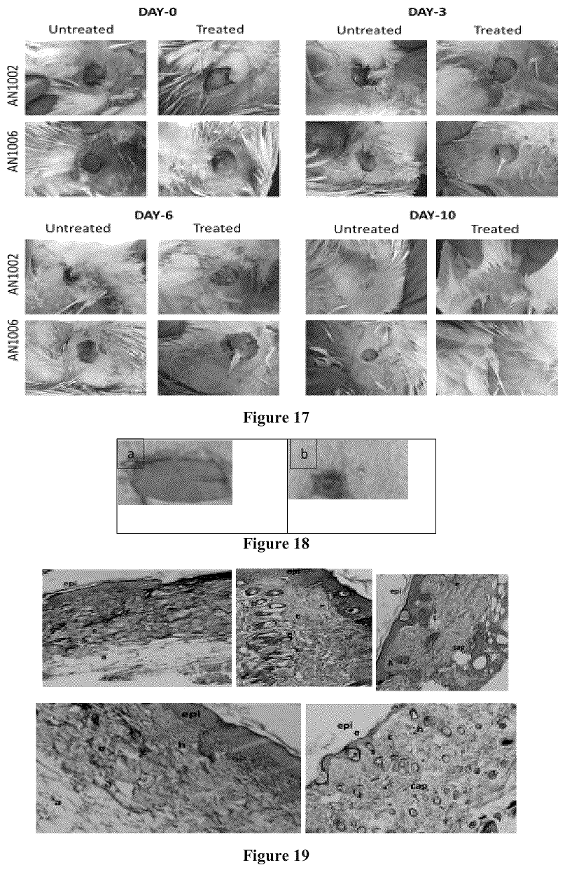

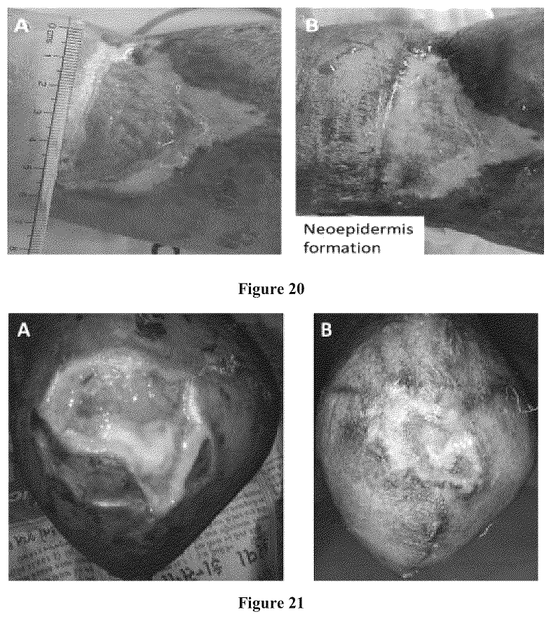

After the 15^{th} and 30^{th} day of the treatment of gel composition on chronic wound, it shows significant difference in wound size as shown in in FIG. 17 and FIG. 18.

1. A therapeutic product comprising gel composition which comprises Lympho-Myeloid Niches cell composition suspended in a biocompatible sterile collagen gel comprises 1-50% cell culture medium, 1-50% plasma or serum, 1-5% antibiotics, and thickener, wherein the supplements cell culture medium, plasma or serum, antibiotics, and thickener do not exceed 50% of the composition.

2. The therapeutic product as claimed in claim 1, wherein the cell culture medium used for growing the cells are selected from MEM, DMEM, F12, DMEM/F12, IMDM, M-199, RPMI medium, serum free medium, T cell medium, stem cell medium, keratinocyte culture medium, cell specific culture medium, normal saline, ringer lactate solution, phosphate buffered solution, balanced salt solution and combination thereof.

3. The therapeutic product as claimed in claim 1, wherein the plasma or serum is selected from the human or animal origin, autologous or allogeneic, purified serum or plasma components.

4. The therapeutic product as claimed in claim 1, wherein the Lympho-Myeloid Nich cell composition comprises macrophages, lymphocytes, dendritic cell, endothelial cells, progenitor cells, and stem cells.

5. The therapeutic product as claimed in claim 1, wherein the thickener is any one of synthetic polymers and natural polymers to prepare gel.

6. The therapeutic product as claimed in claim 1, wherein the thickener is selected from the group consisting of carbohydrate, poly olefinic, pyrrolidone, silicone, and combination thereof.

7. The therapeutic product as claimed in claim 1, wherein the antibiotics used for preparing the cell based therapeutic gel product selected from the group consisting gentamicin, kanamycin, penicillin, streptomycin, doxycycline, tetracycline, ciprofloxacin, amoxicillin, cefuroxime and cefepime.

8. A process for preparing therapeutic product comprising gel composition which comprises the steps of,
a) recovering or isolating peripheral blood mononuclear cells (PBMC) from blood sample collected from a patient through ficoll gradient centrifugation;
b) resuspending the final cell pellet in RPMI medium containing 20% fetal bovine serum as growth medium;
c) counting and seeding PBMC in cell culture flask or vessel;
d) incubating the culture flask or vessel at 37° C. temperature in the presence of 5% CO₂;
e) removing non adherent cells from culture flask or vessel by gently shaking and followed by flushing the adherent cells with the RPMI medium;
f) maintaining the adherent cells in Lympho-Myeloid Niches (LMN), macrophages, lymphocytes, dendritic cell, endothelial cells, progenitor cells, and stem cells for long term by removing the old RPMI medium and then adding fresh RPMI medium once in every 2-3 days;
g) collecting the non-adherent cells generated and detached from the niche by centrifugation and harvesting the adherent cells from the culture using cell dissociating reagents or scraping the cells through cell scraper,
h) suspending the cells obtained from step e) in a biocompatible sterile collagen gel comprising 20% RPMI medium, 10% plasma or serum and 1% antibiotic and thickener wherein the supplements RPMI medium, plasma or serum, antibiotic and thickener should not exceed 50% of the composition, suspending the cells in the supplement and then mixing with the gel to make the final therapeutic product.

9. The process of preparing therapeutic product as claimed in claim 8, wherein the cell dissociating reagents used for collecting adherent cells from the LMN are selected from trypsin, trypsin-EDTA, collagenase, elastase, accutase, dispase and EDTA solutions.

10. A method of treating chronic wound in a subject in need thereof comprising applying therapeutic gel composition to the chronic wound which comprises Lympho-Myeloid Niches cell composition suspended in a biocompatible sterile collagen gel comprises 1-50% cell culture medium, 1-50% plasma or serum, 1-5% antibiotics and thickener, wherein the supplements cell culture medium, plasma or serum, antibiotics and thickener do not exceed 50% of the composition.

11. The method of treating chronic wound as claimed in claim 10, wherein the chronic wound is selected from the group consisting of a chronic wound, non-healing surgical wound, diabetic foot ulcers, burn, an infected tissue or wound, vascular ulcers, arterial ulcers, infarction, necrosis, gangrene, and bed sore.

12-30. (canceled)
